(19) United States
(12) Patent Application Publication
(10) Pub. No.: US 2007/0259935 A1
(43) Pub. Date: Nov. 8, 2007

(54) CRYSTALLINE FORMS OF LETROZOLE AND PROCESSES FOR MAKING THEM

(76) Inventor: Raymond J. H. Westheim, Oss (NL)

Correspondence Address: SYNTHON IP INC, 7130 HERITAGE VILLAGE PLAZA, STE 202, GAINESVILLE, VA, 20155, US

(21) Appl. No.: 11/744,530

(22) Filed: May 4, 2007

Related U.S. Application Data
(60) Provisional application No. 60/797,607, filed on May 4, 2006.

(51) Int. Cl. A61K 31/4196; C07D 249/08

(52) U.S. Cl. 514/383; 548/262.2

(57) ABSTRACT

Crystalline forms of letrozole can be made by precipitation and are useful in making pharmaceutical compositions.

This application claims the benefit of priority under 35 U.S.C. § 119(e) from prior U.S. provisional patent application Ser. No. 60/797,607, filed May 4, 2006, the entire contents of which are incorporated herein by reference.

### BACKGROUND OF THE INVENTION

Letrozole, chemically 4,4′-(H-1,2,4-Triazol-1-ylmethylene)dibenzonitrile of the formula

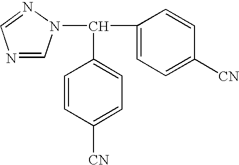

is a known chemical substance used as a pharmaceutically active substance in the pharmaceutical industry. Letrozole is indicated for, inter alia, treatment of advanced breast cancer in postmenopausal women with disease progression following antiestrogen therapy. It is a non-steroidal competitive inhibitor of the aromatase enzyme system.

Letrozole is marketed under the brand name FEMARA® by Novartis Pharmaceuticals as a film coated tablet containing 2.5 mg of letrozole as the free base.

Letrozole is a white to yellowish crystalline powder, practically odorless, freely soluble in dichloromethane, slightly in ethanol, and practically insoluble in water; m. p. 184°-185° C.

U.S. Pat. No. 4,978,672 and EP 236940 disclose letrozole and three general processes for making it. A purportedly improved process has been disclosed in WO 2004/076409.

It is generally known that a solid state chemical compound may often exist in various crystalline forms, differing in the spatial arrangement of molecules in the crystalline lattice. Due to that, such forms exhibit different X-ray powder diffraction (XRPD) patterns and/or IR spectra and are often can have different melting points. More importantly, different crystalline forms may differ in physico-chemical properties such as stability, solubility, etc. and, if the compound serves as a pharmaceutical active substance, those differences may also be reflected in its bioavailability after administration. Usually, only one or a few forms of a chemical compound are stable at normal conditions of storage and the other forms, once obtained, often convert into a thermodynamically more stable form over the course of time.

Despite that the formation of various crystalline forms is known in general, it is not possible to judge in advance whether a specific chemical compound will exist in various crystalline forms or not. Nor, if it does, what the properties of the respective forms will be.

In case of letrozole, no crystalline forms have been described in the prior art documents.

### SUMMARY OF THE INVENTION

The present invention is based on the discovery of two crystalline forms of letrozole and processes for forming them. Accordingly a first aspect of the invention relates to a crystalline letrozole having either (1) XRPD peaks at 2θ of 27.69° and 27.99°, each +/−0.05°; or (2) XRPD peaks at 2θ of 26.42° and 28.10°+/−0.05°. The two peaks are characteristic peaks of Form I and Form II letrozole, respectively. A crystalline letrozole substance having an XRPD that substantially corresponds with FIG. 1 or FIG. 2 is generally preferred and is often referred to herein as letrozole Form I and letrozole Form II, respectively. These letrozole crystal forms can be used in pharmaceutical compositions; e.g. a in combination with at least one pharmaceutically acceptable excipient, however a letrozole substance corresponding to FIG. 1 is generally preferred for pharmaceutical compositions.

Another aspect of the invention relates to a process of making the above letrozole crystalline forms which comprises precipitating the crystalline letrozole from a solution containing letrozole dissolved in a solvent. For making a crystalline letrozole that has an XRPD that substantially corresponds to FIG. 1, the process typically comprises at least one of the following procedures:

a) Crystallization of letrozole from a hot solution in methanol, acetonitrile or tetrahydrofuran by cooling of the optionally stirred solution.

b) Crystallization of letrozole by combining a hot solution in methanol or ethanol with an optionally stirred antisolvent. The antisolvent may be water, C5-C8 aliphatic hydrocarbon such as n-hexane or n-heptane, or an C4-C8 aliphatic ether, preferably diisopropyl ether.

c) Crystallization of letrozole from a hot solution, generally in ethanol, toluene or ethyl acetate, etc., under slow cooling, wherein the cooling rate is 0.5° C./min or less, until at least nucleation is substantially complete and typically down to the room temperature (20-25° C.).

d) Crystallization of letrozole from a solution in chloroform or acetone via a sufficiently slow rate of evaporation of the solvent, and generally at ambient temperature.

For making a crystalline letrozole that has an XRPD that substantially corresponds to FIG. 2, the process typically comprises precipitating the crystalline letrozole from a toluene solution thereof by cooling at a more rapid rate of at least 1° C./min.

In each of these processes, precipitation of the crystalline letrozole can be followed by isolation of the crystalline material from the mother liquor and drying the material until the volatile residuals, if any, are removed.

A further aspect of the invention relates to a process for converting a solid letrozole to Form 1 letrozole, which comprises suspending a solid letrozole substance having an XRPD that does not substantially correspond to FIG. 1 in an organic solvent for a sufficient time to convert said solid letrozole substance to a crystalline letrozole having an XRPD substantially corresponding to FIG. 1.

Another aspect of the invention relates to the use of crystalline letrozole having an XRPD substantially corresponding to FIG. 1 in forming a pharmaceutical composition.

### BRIEF DESCRIPTION OF THE DRAWINGS

FIG. 1 shows the XRPD spectrum of letrozole produced in Example 2 (Form I).

FIG. 2 shows the XRPD spectrum of letrozole produced in Example 1 (Form II).

FIG. 3 shows the XRPD spectrum of letrozole produced in Reference Example 2 (corresponding to Example 26 of U.S. Pat. No. 4,978,672).

FIG. 4 shows an overlay of the portion of the XRPD for each of FIGS. 1-3 for the range of 25°-30° 2θ. The order from top to bottom is FIG. 1, FIG. 3, and FIG. 2.

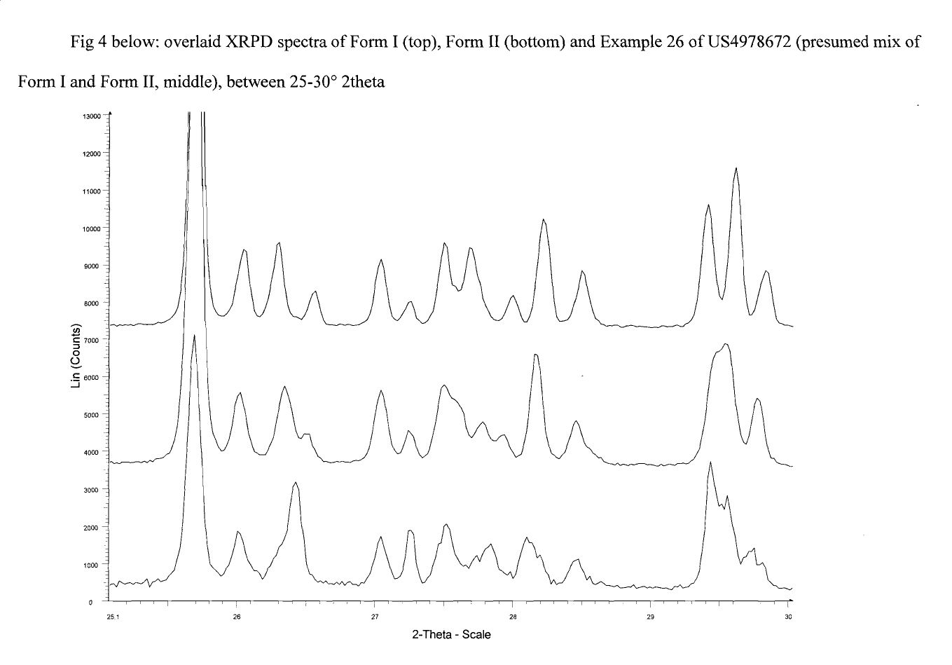

### DETAILED DESCRIPTION OF THE INVENTION

The present invention is based on the discovery of the existence of two crystalline forms of letrozole and processes for making the same. Surprisingly the crystallization techniques as described in U.S. Pat. No. 4,978,672 do not form crystalline letrozole as either Form I or Form II, as described in the present application. Rather these techniques appear to create either (i) a different form, e.g. a Form III, or (ii) a mixture and/or solid solution of Forms I and II.

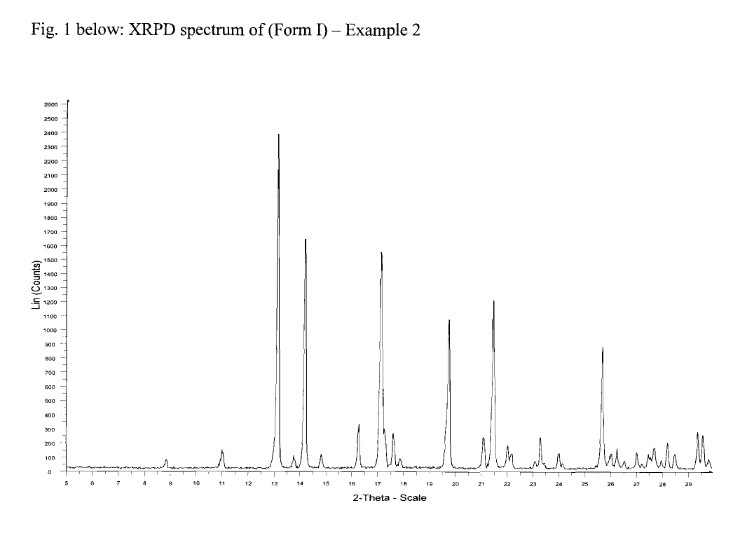

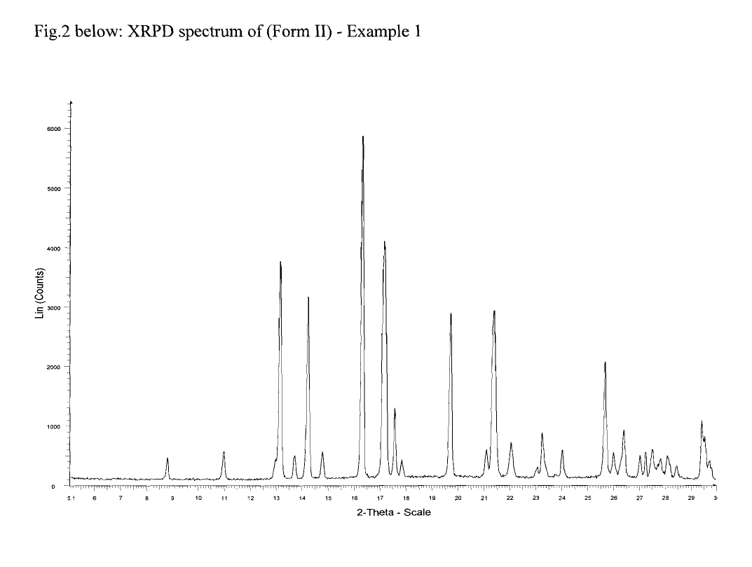

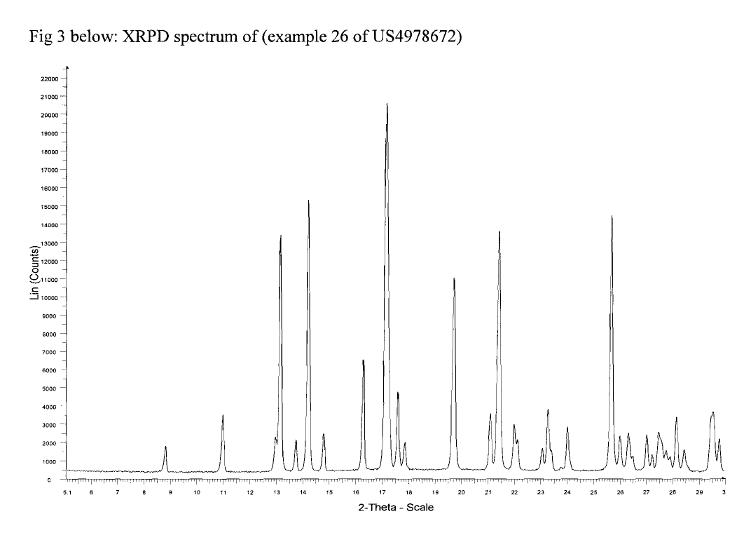

A crystalline letrozole substance that exhibits an XRPD pattern having peaks at 2θ of 27.69° and 27.99°+/−0.05°, or preferably that substantially corresponds with FIG. 1 is considered to be Form I letrozole. A crystalline letrozole substance that exhibits an XRPD pattern having peaks at 2θ of 26.42° and 28.10°+/−0.05°, or preferably that substantially corresponds with FIG. 2 is considered to be Form II letrozole. The phrase “substantially corresponds” is used to allow for variations caused by different sample preparations, different equipment and/or settings used in measuring, normal experimental error/variation and small amounts of impurities. Differences in a pattern that are not attributable to these factors indicate that the letrozole being tested is not Form I (or Form II) letrozole. For example, neither of the letrozole substances that produced FIGS. 2 and 3 “substantially correspond” to FIG. 1 because of the significant differences between the patterns, which were created using the same measuring/analysis technique.

As mentioned above, Form I letrozole can be identified or distinguished by XRPD pattern peaks at 2θ of 27.69° and 27.99°+/−0.05°, and typically Form I can be characterized by including XRPD peaks at 2θ of 21.48°, 22.17°, 23.47°, 26.29°, 26.55°, 27.69°, 27.99°, 28.23°, 28.51° and 29.85°+/−0.05°. For clarity, the designation “+/−0.05” always means that each peak can vary by the stated amount, namely 0.05°.

Form II letrozole can generally be identified or distinguished by XRPD pattern peaks at 2θ of 26.42° and 28.10°+/−0.05°and typically Form II letrozole can be characterized by including XRPD peaks at 2θ of 21.39°, 26.42°, 27.83°, 28.10°, and 29.76°+/−0.05°.

Other physical differences exist between Form I and Form II as can be seen from differences in IR spectra. In particular differences in the IR spectrum in peak intensities are seen at the wavenumbers around 1354-1355 cm⁻¹, 1289-1290 cm⁻¹, 1215-1222 cm⁻¹, 1013-1017 cm⁻¹ and 880-882 cm⁻¹.

Many of the characteristic peaks for the prior art form, e.g. in Examples 25 and 26 of U.S. Pat. No. 4,978,672, are positioned between the values that are characteristic for the Forms I and II. Typically, the characteristic peaks in the XRPD pattern of the replicated prior art crystalline letrozole are positioned at the following angles of 2θ: 21.43°, 26.50°, 27.78°, 27.93°, 28.17°, 29.55° and 29.78°+/−0.05°.

It is questionable whether these positions of XRPD peaks indicate that such a product is actually a mixture of both forms or not. Normally, the XRPD spectrum of a mixture of two crystalline forms would be the sum of the XRPD spectra of the two separate forms. In other words, the peaks of both forms would be visible. Here, instead, the peaks are shifted. There is no unambiguous explanation why peak shifts occur in the present case, instead of the appearance of peaks for both forms. For that reason, one may regard the above product as a third crystalline form (Form III). On the contrary, it has been found that varying the ratios of Form I and II in a deliberate mixture likewise varies the shift in XRPD peak(s), which leads to the belief that the prior art form is in fact a solid solution of the two forms. Thus, a crystalline product comprising a mixture of Form II with a small fraction of Form I would exhibit XRPD peaks characteristic for Form II, but slightly shifted towards the positions of Form I. Conversely, the product comprising Form I with a small fraction of Form II would show peaks for Form I, slightly shifted to positions of Form II. Around 1:1 ratio, the peaks would be between those of Form I and Form II.

Under this concept, crystalline letrozole having an XRPD substantially corresponding to FIG. 1 can be understood to be a relatively pure or homogeneous crystalline form. The same is true for crystalline letrozole having an XRPD substantially corresponding to FIG. 2.

In studying Form I and Form II letrozole, the following observations can be made:

Form II is (partially) converted into Form I when stirred in ethanol at room temperature. It is expected that such a transition also takes place in other solvents. Form I does not convert when stirred in various solvents.

Form II is (partially) converted into form I during annealing above 140° C. Form I does not convert above 140° C.

Slow cooling a solution of letrozole in toluene gave mainly Form I, whereas faster cooling a solution in toluene gave Form II. This indicates that the Form II is the kinetic form, while form I is the thermodynamically stable form.

From these observations it can be concluded that Form II is generally less stable than the Form I and thus Form I would preferably be used as the crystalline form for making pharmaceutical compositions, such as tablets, capsules, etc., by combining the same with one or more pharmaceutically acceptable excipient(s). Indeed, due to the relative instability of Form II, the use of Form II or a mixture of Forms I and II could be liable to conversion during storage to form some or more Form I, respectively. Such a variation in crystalline form is generally undesirable in pharmaceuticals as the different forms can have different properties, e.g. bio-absorption, solubility, degradation, etc. Thus, while the use of either crystalline form in a pharmaceutical composition is contemplated, making the composition from a crystalline letrozole that has an XRPD substantially corresponding to FIG. 1 is preferred.

Form I and Form II letrozole can be formed by precipitating the desired crystalline Form from a solution of letrozole dissolved in a solvent. It has been discovered that various conditions of precipitation can be used to create Form I. In general these conditions can be summarized in four categories. As used herein a “hot” solution means above room temperature, and typically means at least about 50° C. up to the reflux temperature. A “cool” solution means a temperature less than 20° C. and typically in the range of −80° C. to 10° C., unless otherwise specified. When used together in the same context, a hot solution has a higher temperature than a cool solution.

1) Cooling a hot solution of letrozole dissolved in methanol, acetonitrile or tetrahydrofuran to precipitate crystalline letrozole Form I. The hot solution is normally stirred during cooling. Preferably, the concentration in methanol and acetonitrile should be about 30 mg/ml or less, and in tetrahydrofuran about 50 mg/ml or less. The initial solution temperature is preferably the reflux temperature. The cooling rate is not limited in these solvents and is preferably spontaneous cooling. A combination of these solvents may be used as well.

2) Combining a hot solution of letrozole in methanol or ethanol with an antisolvent to precipitate crystalline Form I letrozole. Generally the hot solution of letrozole is added to the antisolvent, which is typically stirred. Preferably the antisolvent is cool, having a temperature of about 20° C. or less, typically in the range of −10° C. to 20° C. The antisolvent can be water, C5-C8 aliphatic hydrocarbon such as n-hexane or n-heptane, or an C4-C8 aliphatic ether, preferably diisopropyl ether.

3) Cooling a hot solution of letrozole at a rate of 0.5° C./min or less until at least nucleation is substantially complete to thereby precipitate crystalline letrozole of Form I. By using slow cooling a variety of solvents can be successfully used to make Form I letrozole. For example, ethanol, toluene and ethyl acetate are suitable solvents when appropriate slow cooling conditions are used. Normally the controlled slow cooling is carried out down to the room temperature (20-25° C.) at which point the product is isolated.

For slow cooling a hot solution in ethanol, the concentration of the hot solution preferably does not exceed 25 mg/ml and a cooling rate of about 17.5° C./hour (=0.3° C./min) or slower is advised. Alternatively, the hot solution may be cooled from, e.g., the reflux temperature to about 50° C. at a rate of about 8° C./hr (=0.133° C./min) and then stirred at 50-52° C. for an additional 2 hours for promoting nucleation. Afterward, there is no need to maintain a specific cooling rate to the final temperature; i.e. having substantially completed nucleation during the 2 hour holding period at around 50° C., the grain growth during the remaining cooling will grow the Form I nuclei and not significantly form letrozole Form II. The technique of controlled cooling only until such substantial completion of nucleation is applicable to other solvents as well and not only ethanol.

For slow cooling a hot solution in ethyl acetate, the concentration of the hot solution preferably does not exceed 50 mg/ml, and a cooling rate of about 26° C./hour (=0.43° C./min) or slower is advised.

For slow cooling a hot solution in toluene, the concentration of the hot solution preferably does not exceed 20 mg/ml, and a cooling rate of about 17.5° C./hour (=0.3° C./min) or slower is advised.

Additional solvents may also be used by adjusting the letrozole concentration, e.g. less concentrated favors Form I, and using a sufficiently slow cooling regime, generally less than 0.5° C./min. The use of faster cooling rates can lead to the formation of a mixture of Form I and II.

4) Evaporating the solvent from a solution of letrozole at a sufficiently slow rate to precipitate crystalline Form I letrozole, wherein the solvent is chloroform or acetone. Crystallization of letrozole Form I from a solution in chloroform or acetone via the slow evaporation of the solvent, preferably at ambient temperature, is a useful method generally for small scale production or experiments. For large scale, however, it is generally a less desirable method than the previous described crystallization techniques.

It should be noted that the above techniques are not mutually exclusive in that two or more conditions can be applied simultaneously. For example, the slow cooling techniques of (3) can be used with the methanol solvent of (1).

In any of the above crystallization procedures, it is also advisable to maintain heating/reflux of the hot solution of letrozole for a sufficient time, typically at least 30 minutes, to remove all possible nuclei before cooling. Furthermore, the nucleation may be induced by adding a small amount of letrozole Form I seed crystals during slow cooling. Interestingly, seeding with Form I crystals did not allow for any significant increase in cooling rate; i.e., seeding with fast cooling did not lead to pure form I, but rather to mixtures.

Isolation of the crystals from the reaction mixture may be performed between 20-50° C. by any conventional technique, such as filtration. It is not expected that prolonged stirring of the mixture or stirring at lower temperatures would raise any problems as the Form I does not convert into Form II even when stirred in a solvent for 2 weeks.

Using a higher concentration of the solution than as stated above may also lead to pure form I, though the danger of formation of the kinetically preferred form II might exist, leading to unacceptably high contamination; e.g., non-Form I crystals.

The isolated wet product typically contains a certain amount of the solvent absorbed in the crystals. A solvent-free product may be obtained by conventional drying, preferably at diminished pressure and at elevated temperature, until essentially all volatile residuals are removed. This can be determined or monitored by conventional “loss on drying” testing. The existence of solvates, at least at the wet product stage, was not specifically studied and cannot be excluded.

Any of the crystallization processes disclosed above may provide for a (pure) Form I of letrozole. These processes may also be repeated or different processes applied sequentially if desired, e.g. for purification from side products (if present), controlling crystal size, crystal purity, etc. For example, Form I letrozole produced by spontaneous precipitation from a methanol solution (condition 1 process) can be dissolved in ethanol to form an ethanolic solution and precipitated via slow cooling (condition 2 process) to form letrozole Form I. The procedure in which the crystallization is induced by an antisolvent is recommended if the formation of crystals having a small size is desired.

Concerning forming Form II letrozole, especially letrozole having an XRPD substantially corresponding to FIG. 2, the precipitation is generally carried out by cooling a toluene solution of letrozole from the reflux temperature to room temperature under a cooling rate of about 1° C./min or higher. In laboratory scale, it may be achieved simply by removing the vessel from the heating appliance, e.g. an oil bath, and allowing the content to cool, under stirring, spontaneously. The product is typically isolated at 20-30° C. by filtration. Other solvents may also be used.

Form I letrozole can also be formed by a solvent mediated transition. Such a process can be advantageous if the undesired crystal form is obtained and/or for deliberately converting from one crystal to another. In general the process comprises suspending a solid letrozole substance, typically a mixture of Forms I and II and/or Form III or is Form II, in an organic solvent, particularly in ethanol, for a time sufficient to convert the letrozole substance into Form I; e.g. having an XRPD substantially corresponding to FIG. 1. Typically the contact time in the solvent is at least 24 hours. The suspension is normally stirred during the contact time. This technique can be used to improve crystal purity, if necessary, such as when a precipitation process intended to produce Form I does not have the appropriate peaks and/or does not substantially correspond to FIG. 1. Alternatively, the technique can be used to obtain Form I via a crystallization process that intentionally produces Form II or Form III; i.e. precipitate a crystal letrozole that has an XRPD not substantially corresponding to FIG. 1 and then convert the letrozole into a crystalline form that does substantially correspond with FIG. 1. Such a process may be more economically depending on the relative precipitation conditions, etc.

The letrozole crystal forms of the present invention can be used in making a pharmaceutical composition. The compositions contain the crystalline letrozole and at least one pharmaceutically acceptable excipient. The compositions can be formed by any suitable process and generally comprise combining the crystalline letrozole with one or pharmaceutically acceptable excipients. The compositions can be solid oral dosage forms such as tablets or capsules. The amount of letrozole, the dosing regimen, and the method of use/treatment in countering breast cancer are generally the same as for the previously known letrozole compositions. Because it is thermodynamically more stable, a crystalline letrozole having XRPD peaks at 27.69° and 27.99°, each +/−0.05° (i.e. Form I), especially having an XRPD that substantially corresponds to FIG. 1, is preferred for use in making the pharmaceutical composition.

The following non-limiting examples further explain the present invention.

### EXAMPLES

### Reference Example 1

Letrozole Prepared According to the Prior Art (Example 25 of U.S. Pat. No. 4,978,672)

1.0 g of letrozole was dissolved in 26 ml of ethanol (containing approximately 5 vol % of water taken up from the air) at reflux and under stirring. Reflux was maintained for about 15 minutes. The hot solution was removed from the oil bath and allowed to cool to R.T., while being stirred. Fast crystallisation already occurred after a few minutes. The solid was isolated by filtration over a P3-glass filter (reduced pressure) and air dried overnight at R.T. and under ambient conditions. A white, crystalline powder was obtained. The yield was 720 mg.

DSC: Single melting peak around 184-185° C.

Capillary: melting between 184.2-184.8° C.

XRPD: Similar to FIG. 3

### Reference Example 2

Letrozole Prepared According to the Prior Art (Example 26 of U.S. Pat. No. 4,978,672)

1.0 g of letrozole was dissolved in 20 ml of ethyl acetate at reflux and by means of stirring. Reflux was maintained for about 15 minutes. The hot solution was removed from the oilbath. To the solution, 30 ml of diethylether was added slowly and in steps of 10 ml. After addition of the third 10 ml, crystallisation started. The inner temperature was about 35° C. Stirring was continued for a few minutes. The solid was isolated by filtration over a P3-glass filter (reduced pressure) and air dried overnight at R.T. and under ambient conditions. A white, crystalline powder was obtained. The yield was 500 mg.

DSC: Single melting peak around 184-185° C.

Capillary: melting between 184.2-184.9° C.

XRPD: See FIG. 3

### Example 1

Letrozole Form II

1.0 g of letrozole was dissolved in 50 ml of toluene at reflux. The clear solution was allowed to cool to R.T. and left at R.T. for about 2.5 hours, during which crystallisation occurred. The crystals were isolated by filtration over a P3-glass filter (reduced pressure) and air dried overnight at R.T. and under ambient. Colourless flakes up to a few mm were obtained. The yield was 740 mg.

DSC: Melting peak around 184-186° C. When magnified between 20-180° C., a shallow exotherm between 120-160° C. can be indicated.

XRPD: See FIG. 2

### Example 2

Preparation of Form I from ethanol

1.0 g of letrozole was dissolved in 40 ml of ethanol at reflux. The hot, stirred solution was slowly cooled down. The first crystals appeared around 54° C. inner temperature, after about 1.5 hours of cooling. Slow cooling was continued to about 35° C., taking one additional hour. Prolonged crystal growth took place. The solid was isolated by filtration over a P3-glass filter (reduced pressure) and air dried overnight at R.T. and under ambient conditions. A white, crystalline powder was obtained. The yield was 660 mg.

DSC: single melting peak around 184-186° C.

XRPD: See FIG. 1

HSM: agglomerates of small prism-like or diamond-like crystals (crystals ≦100 μm, agglomerates ≦200 μm).

### Example 3

Preparation of Form I from methanol

0.5 g of letrozole was dissolved in 15 ml of methanol at reflux. The hot, clear solution was allowed to cool to R.T. After about 15 minutes, slow crystallisation started. The suspension was left at R.T. for a few hours, during which further crystal growth took place. The crystals were isolated by filtration over a P3-glass filter (reduced pressure) and air dried overnight at R.T. and under ambient conditions. Colourless and transparent crystals up to a few mm were obtained. The yield was 280 mg.

DSC: Single melting peak around 184-187° C.

XRPD: Form I

HSM: Well facetted rods with prism-like end-sides.

### Example 4

Preparation of the Form I from chloroform

0.5 g of letrozole was dissolved in 10 ml of chloroform at reflux. The clear solution was allowed to cool to R.T. and left at R.T. in an open flask with cotton wool in the neck for very slow evaporation of solvent. After 4 days the solvent was evaporated, yielding a crystalline residue.

DSC: Single melting peak around 184-186° C.

XRPD: Form I

HSM: No distinctive crystal morphology.

### Example 5

Preparation of the Form I from acetone

0.5 g of letrozole was dissolved in 10 ml of acetone at reflux. The clear solution was allowed to cool to R.T. and left at R.T. in an open flask with cotton wool in the neck for very slow evaporation of solvent. After about 1 day sufficient crystals were formed. The crystals were isolated by filtration over a P3-glass filter (reduced pressure) and air dried at R.T. and under ambient conditions for about 3 days. Large, white to colourless flakes up to about 1 cm were obtained. The yield was 220 mg.

DSC: Single melting peak around 185-186° C.

XRPD: Form I

HSM: Irregular agglomerates of plates and rods. Some prism-like structures are recognisable.

### Example 6

Preparation of Form I from methanol/water

0.5 g of letrozole was dissolved in 15 ml of methanol at reflux. The clear solution was dropwise added to 50 ml of demi-water, stirred at R.T. As a result of this, rapid precipitation took place. The suspension was stirred for a few minutes at R.T. The solid was isolated by filtration over a P3-glass filter (reduced pressure) and air dried overnight at R.T. and under ambient conditions. A white, crystalline powder was obtained. The yield was 390 mg.

DSC: single melting peak around 184-186° C.

XRPD: Form I

HSM: See appendix 5, thin needles, which are sometimes rounded. The needles are typically below 30 μm long.

### Example 7

Preparation of Form I from ethanol/heptane

0.5 g of letrozole was dissolved in 20 ml of ethanol at reflux. The clear solution was dropwise added to 50 ml of cold n-heptane, stirred at −10° C. As a result of this, rapid precipitation took place. The suspension was stirred for a few minutes at R.T. The solid was isolated by filtration over a P3-glass filter (reduced pressure) and air dried overnight at R.T. and under ambient conditions. A white solid, was obtained. The yield was 440 mg.

DSC: single melting peak around 184-185° C.

XRPD: Form I

HSM: tiny prism-like or diamond-like crystals, often rounded. The crystals are below 20 μm in size.

### Example 8

Preparation of Form I from acetonitrile

0.5 g of letrozole was dissolved in 15 ml of acetonitrile at reflux. The clear solution was allowed to cool to R.T. and kept at 4° C. for about 2 hours, during which crystallisation took place. The crystals were isolated by filtration over a P3-glass filter (reduced pressure) and air dried at R.T. and under ambient conditions for about 3 days. White to colourless flakes were obtained. The yield was 170 mg.

DSC: Single melting peak around 184-187° C.

XRPD: Form I

HSM: Diamond-like or block-like crystals, often nicely facetted. The crystals, typically between 100-1000 μm in size, are both isolated and in agglomerates.

### Example 9

Preparation of Form I from methanol/diisopropyl ether

0.5 g of letrozole was dissolved in 20 ml of methanol at reflux. The clear solution was added in about 20 seconds to 80 ml of cold di-isopropylether, stirred at about −78° C. No immediate precipitation took place. The solution was stirred just above the CO₂-ice/acetone bath to allow slow warming up of this solution. As a result of this, slow crystallisation took place. The solid was isolated by filtration over a P3-glass filter (reduced pressure) and air dried overnight at R.T. and under ambient conditions. A white solid/powder was obtained. The yield was 290 mg.

DSC: Single sharp melting peak around 184-185° C.

XRPD: Form I

HSM: Agglomerates or aggregates (40-70 μm) of small, thin rods (smaller than 10 μm).

### Example 10

Crystallization of Form I from tetrahydrofuran

0.5 g of letrozole was dissolved in 10 ml of tetrahydrofuran at reflux. The clear solution was allowed to cool to R.T. and left at R.T. for about 4 days, during which some crystallisation occurred. To increase the yield, the solution was exposed to air for about 5 hours, allowing some evaporation of the solvent and additional nucleation. Then, the solution was kept in a closed flask for an additional one day, during which further crystal growth took place. The crystals were isolated by filtration over a P3-glass filter (reduced pressure) and air dried overnight at R.T. and under ambient. Shiny crystals were obtained. The yield was 400 mg.

DSC: Single melting peak around 185-186° C.

XRPD: Form I

HSM: Prism-like, diamond-like or block-like crystals. The crystals are isolated or agglomerated into larger particles.

### Example 11

Preparation of Form I from toluene

1.0 g of letrozole was dissolved in 50 ml of toluene at reflux. Reflux was maintained for about 30 minutes, while stirring was continued. The hot solution was slowly cooled to 77° C. inner temperature, but no crystallisation occurred. The solution was further cooled to 61° C.; a few crystals appeared. Then, the solution was slowly cooled to about 40° C. taking 2 hours. In the latter cooling step crystal growth took place. The solid was isolated by filtration over a P3-glass filter (reduced pressure) and air dried overnight at R.T. and under ambient conditions. A white, crystalline powder was obtained. The yield was 630 mg.

DSC: Single melting peak around 184-186° C.

XRPD: Form I

HSM: Prism-like or block-like crystals (square shaped or rounded).

### Example 12

Preparation of Form I from ethanol

1.0 g of letrozole was dissolved in 40 ml of ethanol at reflux. Reflux was maintained for 15-30 minutes. The hot, stirred solution was slowly cooled down to 50° C. inner temperature. The first crystals appeared around 52° C. inner temperature, after about 3.5 hours of cooling. The solution was stirred at 50-52° C. for about 2 hours, during which further crystal growth took place. The solid was isolated by filtration over a P3-glass filter (reduced pressure) and air dried at R.T. and under ambient conditions for 3 days. A white, crystalline powder was obtained. The yield was 450 mg.

DSC: Single melting peak around 184-185° C.

XRPD: Form I

HSM: Isolated and agglomerated crystals (block-like, prism-like or diamond-like crystals). The crystals are typically below ≦70 μm in size.

### Example 13

Preparation of Form I from ethyl acetate

1.0 g of letrozole was dissolved in 20 ml of ethyl acetate at reflux. Reflux was maintained for about 1 hour. The hot, stirred solution was slowly cooled down to R.T., taking about 2 hours. Around 30-35° C. inner temperature, crystallisation started. The solid was isolated by filtration over a P3-glass filter (reduced pressure) and air dried at R.T. and under ambient conditions for 4 days. A white, crystalline powder was obtained. The yield was 300 mg.

XRPD: Form I

The DSC values are generally obtained on a Mettler Toledo DSC821e/400, differential scanning calorimeter with a ceramic heat flux sensor, nitrogen purge (50 ml/min), aluminium standard 40 μl with pierced lid, 25-220° C. with 10° C./min. The measurement conditions for the XRPD were as follows: Bruker-AXS D8 Vario, θ/2θ geometry, reflection mode, scintillation detector. Silicon Zero Background sample holder. CuKα1, wavelength=1.54060 Å, primary monochromator. Measurement range: 2°-50° 2θ.

Each of the patents and patent applications mentioned above are incorporated herein by reference. The invention having been described it will be obvious that the same may be varied in many ways and all such modifications are contemplated as being within the scope of the invention as defined by the following claims.

I claim:

1. A crystalline letrozole having either (1) XRPD peaks at 2θ of 27.69° and 27.99°, each +/−0.05°; or (2) XRPD peaks at 2θ of 26.42° and 28.10°+/−0.05°.

2. The crystalline letrozole according to claim 1, wherein said crystalline letrozole has XRPD peaks at 2θ of 21.48°, 22.17°, 23.47°, 26.29°, 26.55°, 27.69°, 27.99°, 28.23°, 28.51° and 29.85°, each +/−0.05°.

3. The crystalline letrozole according to claim 2, wherein said crystalline letrozole has an XRPD substantially corresponding to FIG. 1.

4. The crystalline letrozole according to claim 1, wherein said crystalline letrozole has XRPD peaks at 2θ of 21.39°, 26.42°, 27.83°, 28.10°, and 29.76°, each +/−0.05°.

5. The crystalline letrozole according to claim 4, wherein said crystalline letrozole has an XRPD substantially corresponding to FIG. 2.

6. A pharmaceutical composition, comprising crystalline letrozole according to claim 1 and a pharmaceutically acceptable excipient.

7. The pharmaceutical composition according to claim 6, wherein said letrozole has an XRPD that substantially corresponds to FIG. 1.

8. A process for making crystalline letrozole, which comprises precipitating a crystalline letrozole according to claim 1 from a solution containing letrozole dissolved in a solvent.

9. The process according to claim 8, which comprises:
cooling a hot solution of letrozole dissolved in methanol, acetonitrile or tetrahydrofuran to precipitate crystalline letrozole having an XRPD substantially corresponding to FIG. 1.

10. The process according to claim 8, which comprises:
combining a hot solution of letrozole in methanol or ethanol with an antisolvent to precipitate crystalline letrozole having an XRPD substantially corresponding to FIG. 1.

11. The process according to claim 10, wherein said antisolvent is water, C5-C8 aliphatic hydrocarbon, or an C4-C8 aliphatic ether.

12. The process according to claim 11, wherein said antisolvent is diisopropyl ether.

13. The process according to claim 8, which comprises:
cooling a hot solution of letrozole at a rate of 0.5° C./min or less until at least nucleation is substantially complete to precipitate a crystalline letrozole having an XRPD substantially corresponding to FIG. 1.

14. The process according to claim 13, wherein said rate of cooling is 0.4° C./min or less.

15. The process according to claim 8, which comprises:
evaporating the solvent from a solution of letrozole at a sufficiently slow rate to precipitate a crystalline letrozole having an XRPD substantially corresponding to FIG. 1, wherein said solvent is selected from chloroform and acetone.

16. The process according to claim 8, which comprises cooling said solution at a rate of at least 1° C./min and wherein said solvent is toluene, to precipitate a crystalline letrozole having an XRPD that substantially corresponds to FIG. 2.

17. A process for converting a solid letrozole to Form 1 letrozole, which comprises suspending a solid letrozole substance having an XRPD that does not substantially correspond to FIG. 1 in an organic solvent for a sufficient time to convert said solid letrozole substance to a crystalline letrozole having an XRPD substantially corresponding to FIG. 1.

18. The process according to claim 17, wherein said organic solvent is ethanol and said suspension is stirred for at least 24 hours.

19. A process for making a letrozole pharmaceutical composition, which comprises combining a crystalline letrozole having an XRPD substantially corresponding to FIG. 1 and at least one pharmaceutically acceptable excipient to form a pharmaceutical composition.
